(19) United States
(12) Patent Application Publication
(10) Pub. No.: US 2009/0221824 A1
(43) Pub. Date: Sep. 3, 2009

(54) ENANTIOSELECTIVE PROCESS

(76) Inventors: Paul Howard Briner, Oxfordshire (GB); Matthew Colin Thor Fyfe, Oxfordshire (GB); John Paul Madeley, Oxfordshire (GB); Peter John Murray, Oxfordshire (GB); Martin James Procter, Oxfordshire (GB); Felix Spindler, Mattenstrasse (CH)

Correspondence Address: ELI LILLY & COMPANY, PATENT DIVISION, P.O. BOX 6288, INDIANAPOLIS, IN, 46206-6288, US

(21) Appl. No.: 11/573,471

(22) PCT Filed: Aug. 12, 2005
(86) PCT No.: PCT/GB2005/003175

§ 371 Date: Feb. 9, 2007

(30) Foreign Application Priority Data
Aug. 12, 2004 (GB) 0418046.9

(51) Int. Cl. C07D 405/12; C07D 315/00; C07D 417/12

(52) U.S. Cl. 544/405; 549/427; 548/190; 548/365.7

(57) ABSTRACT

A process for the production of compounds comprising the enantioselective hydrogenation of 2-substituted acrylic acid derivatives.

### BACKGROUND OF THE INVENTION

The present invention is directed to a process for the enantioselective production of compounds. In particular, the invention is directed to a method for the enantioselective hydrogenation of compounds of use in the production of pharmaceutically active compounds, especially compounds which are useful as activators of glucokinase for the treatment of type II diabetes.

Methods for the enantioselective hydrogenation of functionalized olefins, ketones and imines are known in the art. However, the enantioselective hydrogenation of 2-substituted acrylic acids has proved less successful than the enantioselective hydrogenation of other starting materials, such as α-enamides, as the substituent in the 2-position has a considerable impact on the degree of enantioselectivity that can be achieved.

The purity of pharmaceutical compounds to be used for methods of treatment is of paramount importance, thus for example, the synthesis of pharmaceutically active compounds of high enantiomeric purity on a commercial scale is a challenge for the pharmaceutical industry.

International Patent Application PCT/US04/03968 (published after the priority date of the present application) discloses various tri(cyclo) substituted amide compounds which are modulators of glucokinase and are useful in the prophylactic or therapeutic treatment of hyperglycemia and diabetes, in particular type II diabetes. Certain of these compounds, for example (2R)-2-(4-cyclobutanesulfonyl phenyl)-N-pyrazin-2-yl-3-(tetrahydropyran-4-yl)propionamide, (2R)-2-(4-cyclobutanesulfonylphenyl)-N-(1-methyl-1H-pyrazol-3-yl)-3-(tetrahydropyran-4-yl)propionamide, (2R)-2-(4-cyclobutanesulfonylphenyl)-N-(5-fluorothiazol-2-yl)-3-(tetrahydropyran-4-yl)propionamide, (2R)-2-(4-cyclopropanesulfonylphenyl)-N-(5-fluorothiazol-2-yl)-3-(tetrahydropyran-4-yl)propionamide and (2R)-2-(4-cyclopropanesulfonylphenyl)-N-pyrazin-2-yl-3-(tetrahydropyran-4-yl)propionamide, have a chiral center thus there is a need for efficient processes for the production of such compounds of high enantiomeric purity.

PCT/US04/03968 describes the synthesis of such compounds by condensation of the corresponding chiral acid:

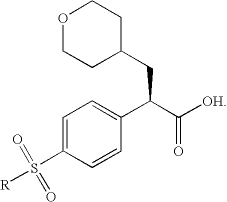

for example where R is cyclobutyl or cyclopropyl, and the corresponding heteroaromatic amine.

The (2R)-propionic acid compound is produced from the corresponding racemic acid i.e. 2-(4-cyclopropanesulfonylphenyl)-3-(tetrahydropyran-4-yl)propionic acid or 2-(4-cyclobutanesulfonylphenyl)-3-(tetrahydropyran-4-yl)propionic acid by condensing with a chiral oxazolidinone derivative to generate a mixture of diastereoisomeric imides that are separable by any conventional method, e.g. column chromatography. Hydrolysis of the pure imides affords the stereopure (2R)-propionic acids that can then be condensed with heterocyclic amines employing a reagent that minimises racemisation of the chiral centre, e.g. benzotriazol-1-yloxytris(pyrrolidino)phosphonium hexafluorophosphate.

However, this process is not particularly efficient for the synthesis of such compounds on a commercial scale. Therefore there is a need for further efficient processes for the production of such compounds of high enantiomeric purity and yield.

### SUMMARY OF THE INVENTION

A process for the production of compounds comprising the enantioselective hydrogenation of 2-substituted acrylic acid derivatives.

### DETAILED DESCRIPTION OF THE INVENTION

The present invention provides a process for the production of a compound of formula (I):

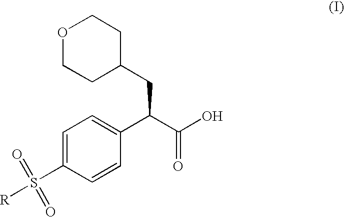

wherein R is cyclopropyl or cyclobutyl, comprising the enantioselective hydrogenation of a compound of formula (II):

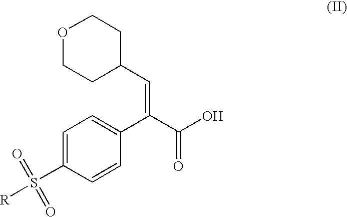

wherein the double bond is in the (E)-configuration and R is cyclopropyl or cyclobutyl.

The hydrogenation of the compounds of formula (II) is preferably conducted in the presence of a rhodium or ruthenium catalyst. The catalyst is preferably an anionic, neutral or cationic rhodium catalyst, more preferably a cationic rhodium catalyst. The catalyst is preferably generated in situ, for example from [Rh(nbd)₂]BF₄, [Rh(nbd)Cl]₂, or [RuI₂(p-cymeme)]₂ and a suitable ligand (nbd=norbornadiene). Alternatively the catalyst may be isolated before use.

Suitable ligands include diphosphine and phosphine ligands, preferably atropisomeric diphosphines, which may have additionally a chiral carbon atom (see M. Scalone Tetrahedron Asymmetry, 1997, 8, 3617; T. Uemura, J. Org. Chem., 1996, 61, 5510; and X. Zhang Synlett, 1994, 501), chiral diphosphine ligands such as for example Josiphos (EP-A-0612758), Walphos (F. Spindler, Adv. Synth. Catal., 2003, 345, 1; EP-A-1 1236994; and U.S. Pat. No. 6,777,567), Phospholane (CH0813/03), Mandyphos (EP-A-0965574; DE-A-1 19921924; and DE-A-1 19956374), Taniaphos (DE-A-1 19952348) and other ferrocene ligands such as for example Jafaphos (EP-A1-803510).

Particularly preferred are ferrocene ligands, for example Mandyphos ligands as described in EP-A-965574. Preferred Mandyphos ligands have the structure:

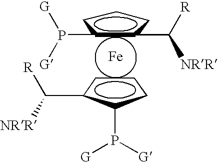

wherein G and G′ which may be the same or different are selected from phenyl optionally substituted with one or more substituents selected from C₁₋₄alkyl and methoxy, e.g. 2-MePh, 3,5-diMePh or 3,5-diMe-4-MeOPh;

R is phenyl or methyl, e.g. phenyl; and

R′ are independently selected from C₁₋₄alkyl, e.g. methyl.

Particular Mandyphos ligands which may be mentioned include (R)—(S)-MOD-Mandyphos and xyl-Mandyphos, especially (R) —(S)-MOD-Mandyphos (structure shown below):

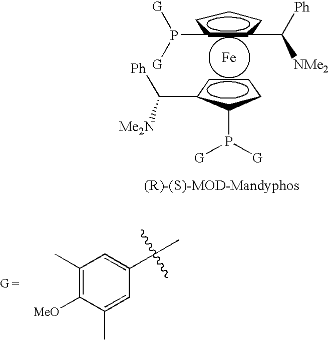

A particularly preferred catalyst/ligand combination is [Rh(nbd)₂]BF₄/(R)—(S)-MOD-Mandyphos.

The hydrogenation pressure used in the process of the invention is preferably up to about 100 bar, such as in the range of 5-100 bar, such as 5-50 bar, for example 15-50 bar.

The temperature used in the process of the invention is preferably up to about 100° C., such as in the range of room temperature to 80° C., e.g. 30-80° C., preferably about 30° C.

The process of the invention is preferably conducted in solution. Suitable solvents include alcoholic solvents, for example methanol, ethanol and iso-propanol. It is particularly preferred that a mixed solvent is used, for example, a mixture of an alcoholic solvent and an arene solvent, e.g. benzene or toluene, preferably toluene, or a mixture of an alcoholic solvent and dimethoxyether, trifluoroethanol or dichloromethane. Suitable mixed solvents include mixtures of alcoholic solvents and toluene, e.g. methanol and toluene. A preferred solvent mixture is 1:1 to 10:1 v/v methanol:toluene, especially about 5:1 v/v methanol:toluene. A further preferred solvent mixture is 1:1 to 10:1 v/v ethanol:toluene, especially about 5:1 v/v ethanol:toluene.

The process of the invention may also be conducted in the presence of base additives e.g. KOH or amines such as NEt₃. A preferred base additive is NEt₃ which may be added to the hydrogenation mixture in an amount of about 10 eq/Rh or Ru.

The (R)-acid of formula (I) may also be further enantiomerically enriched, e.g. by recrystallisation. Suitable solvents for recrystallisation include isopropylacetate, isobutylacetate and ethylacetate, preferably isobutylacetate and mixed solvents such as isobutylacetate or ethylacetate and heptane. A preferred solvent mixture is isobutyl acetate and heptane at a ratio of 20:1 to 1:1 v/v e.g. 9:1 v/v. Other solvents include water:acetic acid e.g. 1:1 v/v, and 1-butanol.

The (R)-acid of formula (I) produced according to the process of the invention preferably has ≧85% ee, more preferably ≧90% ee, even more preferably ≧95% ee, and especially ≧98% ee.

The (E)-acids of formula (II) may be prepared as described in the Examples, or by processes analogous thereto. The (E)-acid of formula (II) used in the method of the invention should be as pure as possible. The (E)-acid of formula (II) is preferably washed with water prior to its use as a substrate in the hydrogenation reaction e.g. to remove any chloride ions which may be present.

The invention also provides the use of the compounds of formula (I) prepared as described above as an intermediate for the manufacture of a compound of formula (III), or a pharmaceutically acceptable salt thereof:

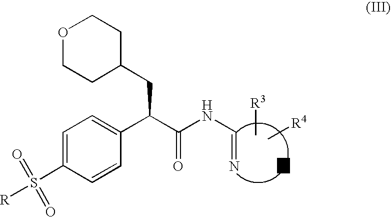

wherein R is cyclopropyl or cyclobutyl;

T together with the —N═C— to which it is attached forms a heteroaryl ring, or a heterocyclic ring where the N═C bond is the only site of unsaturation;

R³ and R⁴ each independently are hydrogen, halogen, OCFₙH₃₋ₙ, methoxy, CO₂R⁵, cyano, nitro, CHO, CONR⁶R⁷, CON(OCH₃)CH₃, or C₁₋₂alkyl, heteroaryl, or C₃₋₇cycloalkyl optionally substituted with 1-5 independent halogen, hydroxy, cyano, methoxy, —NHCO₂CH₃, or —N(C₀₋₂alkyl)(C₀₋₂alkyl) substituents; or R³ and R⁴ together form a 5-8-membered aromatic, heteroaromatic, carbocyclic, or heterocyclic ring;

R⁵ is hydrogen, or a C₁₋₄alkyl group, C₂₋₄alkenyl group, C₂₋₄alkynyl group, C₃₋₇cycloalkyl group, aryl group, heteroaryl group, or 4-7-membered heterocyclic group, wherein any group optionally is substituted with 1-6 independent halogen, cyano, nitro, hydroxy, C₁₋₂alkoxy, —N(C₀₋₂alkyl)(C₀₋₂alkyl), C₁₋₂allyl, C₃₋₇cycloalkyl, 4-7-membered heterocyclic ring, CFₙH₃₋ₙ, aryl, heteroaryl, CO₂H, —COC₁₋₂alkyl, —CON(C₀₋₂alkyl)(C₀₋₂alkyl), SOCH₃, SO₂CH₃, or —SO₂N(C₀₋₂alkyl)(C₀₋₂alkyl) substituents;

R⁶ and R⁷ each independently are hydrogen, or C₁₋₄alkyl group, C₃₋₇cycloalkyl group, aryl group, heteroaryl group, or 4-7-membered heterocyclic group, wherein any group optionally is substituted with 1-6 independent halogen, cyano, nitro, hydroxy, C₁₋₂alkoxy, —N(C₀₋₂alkyl)(C₀₋₂alkyl), C₁₋₂alkyl, C₃₋₇cycloalkyl, 4-7-membered heterocyclic ring, CFₙH₃₋ₙ, aryl, heteroaryl, COC₁₋₂alkyl, —CON(C₀₋₂alkyl)(C₀₋₂alkyl), SOCH₃, SO₂CH₃, or —SO₂N(C₀₋₂alkyl)(C₀₋₂alkyl) substituents; or R⁶ and R⁷ together form a 6-8-membered heterobicyclic ring system or a 4-8-membered heterocyclic ring which optionally is substituted with 1-2 independent C₁₋₂alkyl, CH₂OCH₃, COC₀₋₂alkyl, hydroxy, or SO₂CH₃ substituents; and

n is 1, 2 or 3.

The compounds of formula (III) may be prepared by the condensation of the carboxylic acid of formula (I) with an amine of formula (IV), or a salt thereof:

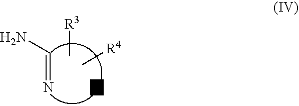

wherein R³ and R⁴ are as defined above, using a variety of coupling conditions, e.g. polymer supported carbodiimide-1-hydroxybenzotriazole in N,N-dimethylformamide at 20° C. (for representative procedures, see http://www.argotech.com/PDF/resins/ps_carbodiimide.pdf and available from Argonaut Technologies, Inc., Foster City, Calif.). Alternatively, a compound of formula (III) may be prepared by condensing amine (IV), or a salt thereof, with an activated acid derivative, such as the corresponding acid chloride. Preferably the condensation is performed employing a reagent that minimises racemisation of the chiral centre, e.g. benzotriazol-1-yloxytris(pyrrolidino)phosphonium hexafluorophosphate (J. Coste et al. Tetrahedron Lett. 1990, 31, 205-208), to furnish enantiopure (R)-amides of formula (III).

Alternatively the coupling reaction may employ an activated derivative of the carboxylic acid of formula (IV), for example a protected ester or acid chloride thereof which may be prepared by methods known to those skilled in the art, in which case the coupling may be conducted in the presence of collidine or another suitable pyridine derivative.

The acid chloride derivatives of the compounds of formula (I) are novel. Thus according to a further aspect of the invention there is provided a compound of formula (V):

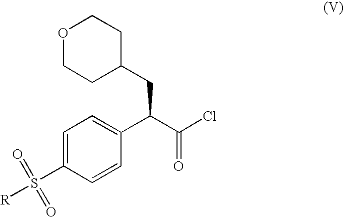

wherein R is cyclopropyl or cyclobutyl.

The acid chlorides of formula (V) may be prepared from the corresponding acids of formula (I) by methods known in the art. The acid chloride may be prepared for example by reaction of the compounds of formula (I) with oxalyl chloride or thionyl chloride in a suitable solvent, such as dichloromethane. The acid chloride may be isolated but is preferably generated in situ prior to coupling with an amine of formula (IV).

Preferred compounds of formula (III) prepared according to this aspect of the invention include those compounds wherein T together with the —N═C— to which it is attached forms a 2-pyrazinyl or 2-thiazolyl ring, and R³ and R⁴ each independently are hydrogen, methyl or fluoro. In particular compounds wherein T together with the —N═C— to which it is attached forms a 5-fluorothiazol-2-yl group. In another embodiment of the invention, compounds of formula (III) include those compounds wherein T together with the —N═C— to which it is attached forms form a 3-pyrazolyl ring, wherein R³ and R⁴ are independently hydrogen or C₁₋₂alkyl, for example a 1-methylpyrazol-3-yl group.

The invention also provides a pharmaceutical composition comprising a compound of formula (III), or a pharmaceutically acceptable salt thereof, produced according to the method described above, in combination with a pharmaceutically acceptable diluent or carrier.

The invention also provides a method of prophylactic or therapeutic treatment of a condition where activation of glucokinase is desirable comprising a step of administering an effective amount of a compound of formula (III), produced according to the method described above, or a pharmaceutically acceptable salt thereof.

The invention also provides a method of prophylactic or therapeutic treatment of hyperglycemia or diabetes, particularly type II diabetes, comprising a step of administering an effective amount of a compound of formula (III), produced according to the method described above, or a pharmaceutically acceptable salt thereof. In this aspect of the invention the compound of formula (III) may be administered in combination with one or more other anti-hyperglycemic agents or anti-diabetic agents.

The invention also provides a method of prevention of diabetes, particularly type II diabetes, in a human demonstrating pre-diabetic hyperglycemia or impaired glucose tolerance comprising a step of administering an effective prophylactic amount of a compound of formula (III), produced according to the method described above, or a pharmaceutically acceptable salt thereof.

All publications, including, but not limited to, patents and patent application cited in this specification, are herein incorporated by reference as if each individual publication were specifically and individually indicated to be incorporated by reference herein as fully set forth.

The invention will now be described by reference to the following examples which are for illustrative purposes and are not to be construed as a limitation of the scope of the present invention.

### EXAMPLES

### Materials and Methods

Column chromatography was carried out on SiO₂ (40-63 mesh). LCMS data were obtained using a Waters Symmetry 3.5μ C₁₈ column (2.1×30.0 mm, flow rate 0.8 mL/min) eluting with solvent A (5% MeCN in H₂O) and solvent B (MeCN solution containing 0.1% HCO₂H) over 6 min and UV detection at 220 nm. Gradient information: 0.0-1.2 min: 100% A; 1.2-3.8 min: Ramp up to 10% A-90% B; 3.8-4.4 min: Hold at 10% A-90% B; 4.4-5.5 min: Ramp up to 100% B; 5.5-6.0 min: Return to 100% A. The mass spectra were obtained employing an electrospray ionisation source in the positive (ES⁺) ion mode. Prep HPLC purification was carried out using a Lunar 10μ ODS2 (250×21.2 mm; flow rate 20 mL/min) eluting with solvent A (0.05% TFA, 10% MeCN, 90% water) and solvent B (0.05% TFA, 90% MeCN, 10% water) and UV detection at 215 nm. Gradient information: 0.0-0.2 min: 90% A, 10% B; 0.2-10.0 min: Ramp up to 10% A, 90% B; 10.0-15.0 min: 10% A, 90% B; 15.0-16.0 min: Return to 90% A, 10% B.

### Preparation 1

### 5-Fluorothiazol-2-ylamine hydrochloride (Method I)

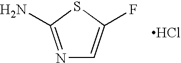

NEt₃ (63.4 mL, 455 mmol) was added to a stirred suspension of 5-bromothiazol-2-ylamine hydrobromide (102.7 g, 379 mmol) in CH₂Cl₂ (1.5 L). After 1 h, TFAA (64.2 mL, 455 mmol) was added dropwise at 0° C. over 15 min. The mixture was allowed to warm to 20° C. over 1 h, before being stirred for an additional 2 h. H₂O (600 mL) was added and the resulting precipitate was collected. The aqueous layer of the filtrate was separated and extracted with CHCl₃ (3×300 mL). The combined organic extracts were washed with brine, dried (Na₂SO₄), filtered and concentrated. The collected precipitate and residual solid were combined and triturated with EtOAc-n-C₆H₁₄ to give N-(5-bromothiazol-2-yl)-2,2,2-trifluoroacetamide: δ_{H} (CDCl₃): 7.45 (1H, s), 13.05 (1H, br). n-BuLi (253 mL of a 1.58M solution in hexanes, 403 mmol) was added dropwise over 50 min to a stirred solution of the above amide (50.0 g, 183 mmol) in anhydrous THF (1.3 L) at −78° C. After 1.5 h, a solution of N-fluorobenzenesulphonimide (86.0 g, 275 mmol) in anhydrous THF (250 mL) was added dropwise over 30 min. The mixture was stirred for 3 h, before being warmed up to −30° C. H₂O (300 mL) was added and the mixture was filtered through a Celite pad. The solid collected and Celite were washed with Et₂₀ (400 mL) and H₂O (400 mL). The organic layer of the filtrate was separated and extracted with water (2×400 mL). The combined aqueous layers were washed with Et₂O (400 mL), before being acidified to pH 6.5 with 2M HCl and extracted with EtOAc (2×400 mL). The combined organic extracts were washed with H₂O (2×400 mL) and brine, before being dried (MgSO₄), filtered and concentrated. Column chromatography (EtOAc-n-C₆H₁₄, 1:3 to 1:2) gave N-(5-fluorothiazol-2-yl)-2,2,2-trifluoroacetamide: δ_{H} (CDCl₃): 7.13 (1H, d). AcCl (12.6 mL, 175 mmol) was added dropwise to a stirred solution of this amide (15.7 g, 73 mmol) in MeOH (300 mL) at 0° C. The mixture was stirred at 20° C. for 30 min, heated under reflux for 1 h, and finally concentrated in vacuo. The residual solid was triturated with THF to give the title compound: δ_{H} (D₂O): 7.00 (1H, d).

### Preparation 2

### 5-Fluorothiazol-2-ylamine hydrochloride (Method II)

### a) 2-(Tert-butoxycarbonylamino)-5-fluorothiazole

2-(Tert-butoxycarbonylamino)thiazole (10 g, 0.050 mol) in THF (0.2 L) was cooled to −50° C. under argon. tBuLi solution in pentane (60 mL of a 1.7M solution, 0.102 mol, 2.05 eq) was added over a period of 30 min and the temperature kept below −40° C. The suspension thus obtained was stirred at −50° C. for 30 min. A solution of N-fluorobenzenesulfonimide (NFSi) was prepared (22.0 g, 0.07 mol in 70 mL THF, 1.4 eq) and 50 mL of this solution (1 eq) was added over a 5 min period and the temperature kept under −40° C. The reaction was stirred for 20 min at −50° C. Then tBuLi (10 mL, 0.017 mol, 0.35 eq) and the NFSi solution (10 mL, 0.4 eq) added. The solution thus obtained was stirred at −50° C. for 45 min and then added to saturated NH₄Cl solution (300 mL). The organic phase was separated and the aqueous phase further washed with diethylether (100 mL). The combined organic fractions were washed with brine (20 mL) solution and dried (Na₂SO₄). The solvent was removed and the solid further dried to afford a brown solid. To this crude product was added trifluoroethanol (60 mL) and formic acid (0.6 mL). The suspension was heated to 85° C. until it gave a solution. The flask was then cooled to RT and the precipitate thus formed filtered off to afford, after drying under high vacuum, the title compound (6.4 g, contains 2.3% of starting material according to HPLC at 275 nm). After a second crystallisation (trifluoroethanol (22 mL) and formic acid (0.22 mL) for 20 min at 85° C.), the title compound was obtained as an off white solid (4.6 g, contains 1% of starting material, 97.5% pure by HPLC). ¹H NMR (CDCl₃, 300 MHz) δ: 6.90 (1H, d, CHCF), 1.60 (9H, s, Boc-H).

### b) 5-Fluorothiazol-2-ylamine hydrochloride

2-(Tert-butoxycarbonylamino)-5-fluorothiazole (4.6 g, 21.1 mmol) was dissolved in dioxane (25 mL). HCl gas was bubbled through the solution for 4 h, then diethyl ether (50 mL) was added to give a suspension which was filtered off. The solid was dried in high vacuum to afford the title compound (3.03 g, 19.7 mmol, 93% yield). ¹H NMR (D₂O) δ: 7.00 (1H, d); m/z=119.0 [M+ H]⁺.

### Preparation 3

### Ethyl (4-cyclopropylsulfanylphenyl)oxoacetate

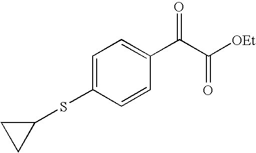

AlCl₃ (104.6 g, 0.79 mol) was suspended in CH₂Cl₂ (1.15 L) and cooled in an ice/salt bath to 0° C. with stirring. Ethyl chlorooxoacetate (84.8 g, 0.62 mol) was then added over a period of 10 min, during which time the temperature was maintained between 0 and 2° C. The mixture was then stirred for a further 30 min at 0° C., before the addition of cyclopropylphenylsulfide (85.0 g, 0.57 mol) over a period of 45 min, during which time the temperature remained between 0 and 8° C. The resulting mixture was allowed to warm to room temperature and stirred for a further 2 h. After this time ice/water (275 mL) was added, with ice bath cooling maintaining the temperature at 20° C. The organic layer was separated and washed with water (2×250 mL), saturated NaHCO₃ solution (2×250 mL) and again with water (1×250 mL). The organic fraction was then dried (MgSO₄) filtered and the solvent removed to provide the title compound (134 g, 94% yield). NMR was consistent with the above structure.

### Preparation 4

### Ethyl (4-cyclopropylsulfonylphenyl)oxoacetate

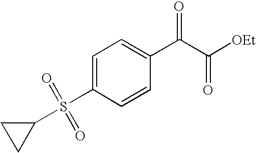

To a stirred solution of Preparation 3 (49.4 g, 0.2 mol) in CH₂Cl₂ (180 mL) was added a solution of m-chloroperoxybenzoic acid (92.0 g, 0.40 mol, calc as 75% strength) in CH₂Cl₂ (650 mL) over 45 min with the temperature maintained at 15-25° C. TLC (CH₂Cl₂:ethyl acetate 1:10) showed that starting material still remained. Further m-chloroperoxybenzoic acid (3.4 g) in CH₂Cl₂ was added and the reaction stirred for 30 min. A second TLC still showed the presence of some starting material, and additional m-chloroperoxybenzoic acid (3.4 g) was added and the reaction stirred for a further 2 h. TLC showed a small amount of starting material so a final quantity of m-chloroperoxybenzoic acid (11.0 g) was added and the reaction continued for 1 h. Sodium carbonate solution (2M, 500 ml) was then added and the aqueous layer was separated, the pH raised to 9-10 and reextracted with CH₂Cl₂. The organic extracts were combined, washed with water (2×400 ml), dried (MgSO₄), filtered and the solvent removed under vacuum (54.1 g, 96% yield). NMR was consistent with the above structure.

### Preparation 5

### (Tetrahydropyran-4-yl)methanol

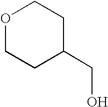

To a suspension of LiAlH₄ (56 g, 1.47 mol) in diethyl ether (2 L) under argon was added methyl tetrahydro-2H-pyran-4-carboxylate (270 g, 1.88 mol) in diethyl ether (ca. 200 mL) under reflux over a period of 1.75 h. After addition was complete reflux was continued for a further 1 h. TLC (diethyl ether) indicated a small amount of ester remained, so further LiAlH₄ (10 g, 0.26 mol) was added and reflux continued for 1 h. Water (66 mL) was added, then 15% NaOH solution (66 mL), followed by further water (198 mL). The solid was filtered and dried to give the crude product, which was redissolved in DCM (800 ml), dried (MgSO₄), filtered and the solvent removed to afford the title compound (207 g, 94% yield). NMR was consistent with the above structure.

### Preparation 6

### Methanesulfonic acid (tetrahydropyran-4-yl)methyl ester

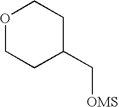

To a mixture of Preparation 5 (216.5 g, 1.87 mol) and triethylamine (299 mL) in DCM (1.3 L) at <10° C. was added under argon a solution of methanesulfonyl chloride (236 g, 160 mL) in DCM (200 mL) over 2 h 50 min, maintaining the temperature at 5-10° C. throughout. Subsequent washing with water (1 L), 1M HCl (500 mL), 5% NaHCO₃ (300 mL), water (300 mL), drying (MgSO₄) and then removal of the solvent afforded the title compound (328 g, 90% yield). NMR was consistent with the above structure.

### Preparation 7

### 4-Iodomethyltetrahydropyran

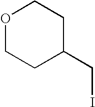

A mixture of Preparation 6 (328 g, 1.69 mol) and sodium iodide (507 g, 3.4 mol) in acetone (3.3 L) was refluxed for 4 h. TLC (diethyl ether) showed significant mesylate remaining so further sodium iodide (127 g, 0.65 mol) was added and reflux continued for 16 h. The mixture was cooled and filtered. The resulting cake was washed with acetone, dried, and then partitioned between diethyl ether (800 mL) and water (800 mL). The aqueous phase was re-extracted with diethyl ether (200 mL), the ether extracts combined and washed with 10% sodium thiosulphate solution (300 mL) which decolourised the extract. Final washing with water (300 mL), drying (MgSO₄) and then removal of the solvent provided the title compound (365 g, 92% yield). NMR was consistent with the above structure.

### Preparation 8

### Triphenyl(tetrahydropyran-4-ylmethyl)phosphonium iodide

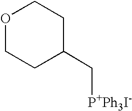

A mixture of Preparation 7 (350 g, 1.55M) and triphenylphosphine (406 g, 1.55M) in acetonitrile (1.6 L) was heated under reflux. After 27 h the mixture was cooled and filtered, washed with diethyl ether and dried in air to provide a white solid (504 g). Filtrate and washings were returned to reflux and concentrated to 750 mL, reflux was maintained for 16 h before cooling and dilution with diethyl ether (ca 1.2 L). A precipitate formed which was stirred for 30 min before being filtered, washed with diethyl ether (2×300 mL) and dried in air to yield a further crop (100 g). Overall yield of the title compound (604 g, 80%). RT=2.7 min; m/z (ES⁺)=361.2.

### Preparation 9

### (E)-2-(4-Cyclopropanesulfonylphenyl)-3-(tetrahydropyran-4-yl)acrylic acid

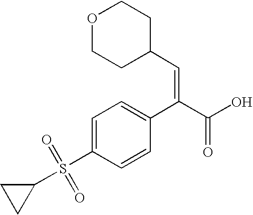

To a suspension of Preparation 8 (2.49 kg, 5.10 mol) in dry THF (5 L) maintained between −5 and 0° C. was added a solution of lithium hexamethyldisilazide (1.05M, 4.39 kg, 5.18 mol) over 30 min. The resulting mixture was then warmed to 15° C. and stirred for 2 h before recooling to between 0 and 5° C. A solution of Preparation 4 (1.25 kg, 4.43 mol) in THF (2.5 L) was then added over 1 h, during which time the temperature was maintained between 0 and 5° C., before a period of 16 h at between 20 and 25° C. Subsequently, brine (17% w/w, 3.8 L) was added and the phases separated with the aid of additional brine (1.3 L). The aqueous phase was reextracted with methyl t-butyl ether (2×2.5 L) and the combined organic extracts washed with brine (2×3.8 L). The solvents were removed under vacuum at between 30 and 40° C. The residue was dissolved in methanol (15 L) and aqueous sodium hydroxide (2M, 4.34 L) added before heating at 65-67° C. for 4 h. The mixture was cooled and the solvents removed under vacuum at between 35 and 40° C. until water started to distil. The residue was diluted with water (15 L). The solid phosphine oxide was filtered off, washed with water (2.5 L) and the filtrate separated. The aqueous phase was washed with methyl t-butyl ether (5 L and 3.5 L), before acidification with hydrochloric acid solution (5M, 1.9 L) in the presence of methyl t-butyl ether (10 L). The organic phase was separated and the aqueous phase reextracted with methyl t-butyl ether (5 L). The combined organic extracts were washed with saturated brine (2×1 L) and the solvent removed under vacuum. Methanol (2 L) was added and then removed under vacuum, this step was then repeated. The residue was brought to a total weight of 4.0 kg by addition of methanol and stirred at ambient temperature to crystallise the product. Filtration of the solid and washing with chilled (ca 0° C.) methanol (500 mL) gave, after vacuum drying at 40° C., the title compound (654 g, 41% yield after correction for residual solvent). NMR was consistent with the above structure.

### Example 1

### Preparation of (2R)-2-(4-cyclopropanesulfonylphenyl)-3-(tetrahydropyran-4-yl)propionic acid

(E)-2-(4-Cyclopropanesulfonylphenyl)-3-(tetrahydropyran-4-yl)acrylic acid (Preparation 9, 110 g, 0.327 mol) was dissolved in MeOH/Toluene 5:1 (1.4 L). In a 4.0 mL Schlenk flask was placed [Rh(nbd)₂](BF₄) (30.5 mg, 0.08 mmol) and All-MOD-Mandyphos (90.4 mg, 0.08 mmol), dissolved in MeOH (10 mL) and stirred for 1 h at RT. This catalyst solution was then added to the (E)-2-(4-cyclopropanesulfonylphenyl)-3-(tetrahydropyran-4-yl)acrylic acid solution and transferred to a 2.5 L autoclave. The autoclave was pressurized to 50 bar and heated to 30° C. After 18 h the pressure was released and the solution transferred to a 3 L flask. Active charcoal (3 g) was added to the reaction mixture, stirred for 1 h and the charcoal removed by filtration. The solution was further filtered over Hyflo and a Zeta-Bond filter. The solution thus obtained was concentrated under partial pressure and the solid obtained further dried under high vacuum to give a solid (105 g). The solid was placed in a 1.5 L flask equipped with a mechanical stirrer, a thermometer and a dropping funnel. Isobutylacetate (540 mL) was added at RT and the suspension heated at 110° C. until a clear solution was observed. Heptane (60 mL) was added slowly at 110° C., the oil bath was then removed and the solution allowed to cool slowly. The reaction was stirred for a further 16 h, the title compound filtered off and dried under high vacuum (77.2 g, 70% yield, 99% ee). ¹H NMR (CDCl₃, 300.13 MHz) δ: 7.85 (2H, Aryl H, d, J_{HH}=6.6 Hz), 7.50 (2H, Aryl H, d, J_{HH}=6.6 Hz), 3.95 (br d, 2H), 3.80 (t, 1H, CHCH₂, J_{HH}=7.8 Hz), 3.35 (m, 2H), 2.45 (m, 1H), 2.10 (m, 1H), 1.75 (m, 1H), 1.60 (m, 2H), 1.50-1.20 (m, 5H), 1.05 (m, 2H).

### Example 2

### Preparation of (2R)-2-(4-cyclopropanesulfonylphenyl)-N-(5-fluorothiazol-2-yl)-3-(tetrahydropyran-4-yl)propionamide

A mixture of CH₂Cl₂ (1.35 L) and DMF (35.9 mL, 0.465 mol, 1.5 eq) was cooled to −20° C. and oxalylchloride (39.4 mL) slowly added (0.465 mol, 1.5 eq). After stirring for 45 min (2R)-2-(4-cyclopropanesulfonylphenyl)-3-(tetrahydropyran-4-yl)propionic acid (Example 1, 105.0 g, 0.310 mol, 1 eq) was added. The reaction was stirred at −20° C. for 1 h. Collidine (185 mL, 1.395 mol, 4.5 eq) was then slowly added. The reaction mixture was stirred for 15 min and then 5-fluorothiazol-2-ylamine hydrochloride (Preparation 1, 52.7 g, 0.341 mol, 1.1 eq) was added at −15° C. The resulting suspension was kept at −15° C. for 2 h after which the ice bath was removed and the reaction slowly warmed to RT over a period of 2 h. The mixture was evaporated to dryness to afford a semi-solid to which was added portionwise 4N HCl solution (1.5 mL). The product was extracted with ethylacetate (3 L) and the organic fraction further washed with water (1 L) and saturated NaHCO₃ solution (1 L). The solvent was removed under partial vacuum to give the title compound (135 g). Characterising data was consistent with the formation of the title compound.

### Example 3

### Preparation of (2R)-2-(4-cyclopropanesulfonylphenyl)-N-pyrazin-2-yl-3-(tetrahydropyran-4-yl)propionamide

In a 10 L reaction vessel purged with N2 were placed dichloromethane (2 L) and N,N-dimethylformamide (54.7 mL, 0.709 mol). The solution was cooled to −10° C. and oxalylchloride (60.0 mL, 0.709 mol) slowly added over 15 min resulting into strong gas evolution. The white suspension which formed was stirred until no more gas evolved then cooled to −20° C. A suspension of (2R)-2-(4-cyclopropanesulfonylphenyl)-3-(tetrahydropyran-4-yl)propionic acid (Example 1, 200 g, 0.590 mol) in dichloromethane (1 L) was added over 1 h. The yellow solution was stirred for a further 0.5 h, then cooled to −45° C. and a solution of pyridine (51.0 mL, 0.590 mol) in dichloromethane (0.25 L) added over 20 min. While the temperature was kept under −45° C., a suspension of 2-aminopyrazine (112.4 g, 1.180 mol) in THF (1.1 L) and a solution of pyridine (154 mL, 1.770 mol) in dichloromethane (0.9 L) were added in parallel over 1.25 h. The cooling bath was removed and the orange suspension was stirred for a further 16 h. The solution was concentrated under vacuum by means of a rotavap (250 mbar, 50° C.) and 3×800 mL portion of ethyl acetate were added and further distilled off. To the brown oil were added ethyl acetate (2 L) and aqueous HCl (2M, 2 L). The phases were separated and the organic phase further washed with aqueous HCl (2M, 2×1 L). The aqueous phase were collected and washed with ethyl acetate (2 L). The organic phases were collected and washed with water (2 L) and a saturated solution of NaHCO₃ (2×1 L). The HPLC analysis of the basic aqueous phase showed it contained some product and therefore was extracted with ethyl acetate (2 L). The organic fractions were collected and washed with water (1 L) and brine (1 L). The organic phase was dried over Na₂SO₄ and the suspension filtered off over a paper filter. The solvent was removed by means of a rotavap (125 mbar, 50° C.) and ethanol was added portionwise (3×0.5 L) to ensure complete removal of the ethyl acetate for the next step. A brown oil was isolated (248 g) which according to HPLC and ¹H NMR analyses was 96.5% pure and contained 16% of ethanol (% ee> 98.0%). Corrected yield: 207.3 g (84%). Characterising data was consistent with the formation of the title compound.

### Example 4

### Preparation of (2R)-2-(4-cyclobutanesulfonylphenyl)-3-(tetrahydropyran-4-yl)propionic acid

In a 300 mL autoclave were placed (E)-2-(4-cyclobutanesulfonylphenyl)-3-(tetrahydropyran-4-yl)acrylic acid (5.4 g, 15.5 mmol, prepared by a process analogous to Preparation 9 and that described in PCT/US04/03968), and of MeOH/Toluene 5:1 solution (170 mL). The autoclave was closed and evacuated by means of 10 cycles of vacuum/N2 (10 bar). In a separate Schlenk flask are placed and degassed of MeOH/Toluene 5:1 solution (10 mL). Then the ligand (R)—(S)-MOD-Mandyphos (34.75 mg, 0.033 mmol) and [Rh(nbd)₂](BF₄) (11.6 mg, 0.031 mmol) were added to the Schlenk flask and the apparatus degassed by means of 5 cycles of vacuum/N2. The thus prepared catalyst solution was transferred in the autoclave and 50 bar of H₂ pressure were applied. The reaction was stirred over night. The pressure was released. The solution was evaporated to dryness by means of a rotavap to afford a beige solid (4.5 g, 88% ee (R)).

In a 250 mL reaction vessel were added the crude product and isobutylacetate (80 mL). The suspension was heated at 110° C. until a clear solution was observed. Then heptane (8 mL) was added at 110° C. The oil bath was then removed and the solution cooled down slowly. After standing over night, the precipitate was filtered off and the light beige crystalline solid dried under vacuum (50° C., 200 mbar). After an IPC (HPLC: 93% ee) the crude product was dissolved again in isobutylacetate (80 mL) at 110° C. Once the solution was clear, heptane (8 mL) was added. The oil bath was then removed and the solution cooled down slowly. After standing over night, the precipitate was filtered off and the light beige crystalline solid dried under vacuum (50° C., 200 mbar). This afforded the title compound (3.8 g). Characterising data was consistent with the formation of the title compound.

### Example 5

### Preparation of (2N)-2-(4-cyclobutanesulfonylphenyl)-N-(1-methyl-1H-pyrazol-3-yl)-3-(tetrahydropyran-4-yl)propionamide

In a 100 mL three neck round bottom flask were placed CH₂Cl₂ (40 mL) and (2R)-2-(4-cyclobutanesulfonylphenyl)-3-(tetrahydropyran-4-yl)propionic acid (Example 4, 1.2 g, 3.4 mmol). The solution was cooled to −20° C. before DMF (0.273 g, 3.74 mmol, 1.1 eq.) and oxalylchloride (0.32 g, 3.74 mmol, 1.1 eq) were added. The reaction was further stirred at −20° C. for 1 h to afford a slightly yellow solution. 1-Methyl-1H-3-aminopyrazole (0.99 g, 10.2 mmol, 3 eq) was added at −20° C., then 0.55 g pyridine (0.55 g, 6.8 mmol, 2 eq) was added over the course of 10 min. After stirring at −20° C. for 45 min an IPC was taken. No more acid chloride was present. The reaction solution was warmed up to room temperature before taking up in ethylacetate (100 mL) and washing twice with HCl (1M, 100 mL) followed by washing twice with saturated NaHCO₃. The organic phase was dried over Na₂SO₄ before the solvent was removed. Crude product 1.2 g (82%); HPLC purity: >98%; Enantioselectivity: 98%. Characterising data was consistent with the formation of the title compound.

1. A process for the production of a compound of formula (I): 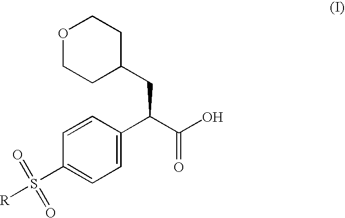
wherein R is cyclopropyl or cyclobutyl, comprising the enantioselective hydrogenation of a compound of formula (II): 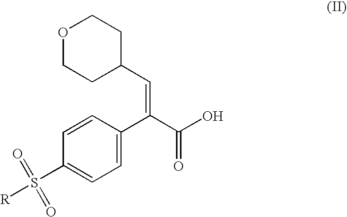
wherein the double bond is in the (E)-configuration and R is cyclopropyl or cyclobutyl.

2. The process according to claim 1 which is conducted in the presence of a rhodium or ruthenium catalyst.

3. The process according to claim 2 wherein the catalyst is generated in situ.

4. The process according to claim 2 wherein the catalyst is generated from [Rh(nbd)₂]BF₄, [Rh(nbd)Cl]₂, or [RuI₂(p-cymene)]₂ and a suitable ligand.

5. The process according to claim 4 wherein the ligand is selected from atropisomeric diphosphine ligands; which may have additionally a chiral carbon atom; chiral diphosphine ligands; and other ferrocene ligands.

6. The process according to claim 5 wherein the ligand is (R)—(S)-MOD-Mandyphos.

7. The process according to claim 6 comprising a catalyst/ligand combination of [Rh(nbd)₂]BF₄/(R)—(S)-MOD-Mandyphos.

8. The process according to claim 1 wherein the hydrogenation is performed at a pressure in the range 15-100 bar.

9. The process according to claim 1 wherein the hydrogenation is conducted at a temperature of from 30 to 80° C.

10. The process according to claim 1 wherein the hydrogenation is conducted in solution.

11. The process according to claim 10 wherein the solvent is an alcoholic solvent.

12. The process according to claim 11 wherein the solvent comprises one of methanol ethanol or iso-propanol.

13. The process according to claim 12 wherein the solvent, also comprises toluene.

14. The process according to claim 13 wherein the solvent is a 1:1 to 10:1 v/v mixture of methanol and toluene or ethanol and toluene.

15. The process according to claim 1 providing the (R)-acid of formula (I) which is further enantiomerically enriched by recrystallisation.

16. The process according to claim 15 wherein the recrystallisation solvent is a mixture of isobutyl acetate and heptane at a ratio of 20:1 to 1:1 v/v.

17. A compound of formula (V): 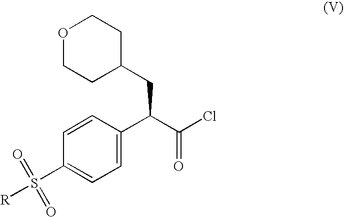
wherein R is cyclopropyl or cyclobutyl.

18. A process for the production of a compound of formula (III), or a pharmaceutically acceptable salt thereof: 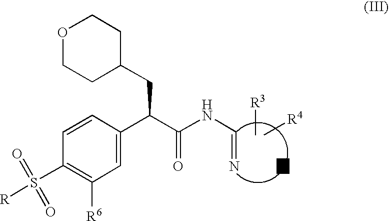
wherein R is cyclopropyl or cyclobutyl;
T together with the —N═C— to which it is attached forms a heteroaryl ring, or a heterocyclic ring where the N═C bond is the only site of unsaturation;
R³ and R⁴ each independently are hydrogen, halogen, OCFₙH₃₋ₙ, methoxy, CO₂R⁵, cyano, nitro, CHO, CONR⁶R⁷, CON(OCH₃)CH₃, or C₁₋₂alkyl, heteroaryl, or C₃₋₇cycloalkyl optionally substituted with 1-5 independent halogen, hydroxy, cyano, methoxy, —NHCO₂CH₃, or —N(C₀₋₂alkyl)(C₀₋₂alkyl) substituents; or R³ and R⁴ together form a 5-8-membered aromatic, heteroaromatic, carbocyclic, or heterocyclic ring;
R⁵ is hydrogen, or a C₁₋₄alkyl group, C₂₋₄alkenyl group, C₂₋₄alkynyl group, C₃₋₇cycloalkyl group, aryl group, heteroaryl group, or 4-7-membered heterocyclic group, wherein any group optionally is substituted with 1-6 substituents independently selected from: halogen, cyano, nitro, hydroxy, C₁₋₂alkoxy, —N(C₀₋₂alkyl)(C₀₋₂alkyl), C₁₋₂alkyl, C₃₋₇cycloalkyl, 4-7-membered heterocyclic ring, CFₙH₃₋ₙ, aryl, heteroaryl, CO₂H, —COC₁₋₂alkyl, —CON(C₀₋₂alkyl)(C₀₋₂alkyl), SOCH₃, SO₂CH₃, or —SO₂N(C₀₋₂alkyl)(C₀₋₂alkyl) substituents;
R⁶ and R⁷ each independently are hydrogen, or C₁₋₄alkyl group, C₃₋₇cycloalkyl group, aryl group, heteroaryl group, or 4-7-membered heterocyclic group, wherein any group optionally is substituted with 1-6 substituents independently selected from: halogen, cyano, nitro, hydroxy, C₁₋₂alkoxy, —N(C₀₋₂alkyl)(C₀₋₂alkyl), C₁₋₂alkyl, C₃₋₇cycloalkyl, 4-7-membered heterocyclic ring, CFₙH₃₋ₙ, aryl, heteroaryl, COC₁₋₂alkyl, —CON(C₀₋₂alkyl)(C₀₋₂alkyl), SOCH₃, SO₂CH₃, or —SO₂N(C₀₋₂alkyl)(C₀₋₂alkyl) substituents; or R⁶ and R⁷ together form a 6-8-membered heterobicyclic ring system or a 4-8-membered heterocyclic ring which optionally is substituted with 1-2 substituents independently selected from: C₁₋₂alkyl, CH₂OCH₃, COC₀₋₂alkyl, hydroxy, or SO₂CH₃ substituents; and
n is 1, 2 or 3;
comprising condensation of a compound of formula (I) produced according to claim 1, or an activated derivative thereof, with an amine of formula (IV), or a salt thereof: 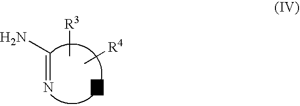
wherein T, R³ and R⁴ are as defined above.

19. The process according to claim 18 wherein T together with the —N═C— to which it is attached forms a 3-pyrazolyl, 2-pyrazinyl or 2-thiazolyl ring, and R³ and R⁴ each independently are hydrogen, methyl or fluoro.

20. The process according to claim 19 wherein T together with the —N═C— to which it is attached forms a 5-fluorothiazol-2-yl group.

21. The process according to claim 19 wherein T together with the —N═C— to which it is attached forms a 1-methyl-1H-pyrazol-3-yl ring.

22. The process according to claim 19 wherein T together with the —N═C— to which it is attached forms a 2-pyrazinyl ring.

23-26. (canceled)
